(19) United States
(12) Patent Application Publication
(10) Pub. No.: US 2022/0290095 A1
(43) Pub. Date: Sep. 15, 2022

(54) Bifidobacterium breve CCFM1025, Food Fermented Thereby and Application Thereof

(71) Applicant: Jiangnan University, Wuxi (CN)

(72) Inventors: Wei CHEN, Wuxi (CN); Gang WANG, Wuxi (CN); Peijun TIAN, Wuxi (CN); Jianxin ZHAO, Wuxi (CN); Hao ZHANG, Wuxi (CN)

(21) Appl. No.: 17/623,936

(22) PCT Filed: Aug. 22, 2018
(86) PCT No.: PCT/CN2018/101685

§ 371 Date: Dec. 30, 2021

(51) Int. Cl. C12N 1/20; A61K 35/745; A61P 25/24; A61P 3/04; A61P 1/00; A23L 33/00; A23L 33/135

(52) U.S. Cl. CPC C12N 1/205; A61K 35/745; A61P 25/24; A61P 3/04; A61P 1/00; A23L 33/40; A23L 33/135; C12R 2001/01

(57) ABSTRACT

Bifidobacterium breve CCFM1025, foods fermented thereby and an application thereof in the preparation of an anti-depression drug, an anti-inflammatory bowel disease drug, an anti-obesity drug, health products and functional foods.

### TECHNICAL FIELD

The disclosure relates to B. breve CCFM1025, food fermented thereby and application thereof, and belongs to the technical field of microbiology.

### BACKGROUND

Depression is also called depressive disorder, including major depressive disorder, bipolar affective disorder, seasonal affective disorder, postpartum depression, etc. As a heterogeneous disease, the pathogenesis of depression has not yet been clearly elucidated. The inconsistency in the response of depression patients to treatment makes depression an increasingly serious global disease burden. There are currently more than 350 million people suffering from depression in the world, and it is estimated that by 2030, depression will become the primary disease in the world.

Currently, there are two major explanations for the mechanism of depression, including monoamine neurotransmitter disorders in the brain and abnormal function of the hypothalamic-pituitary-adrenal axis (HPA). Among them, monoamine neurotransmitter disorders are the main research direction, which have also been confirmed by a large number of cases, and are mainly manifested as decrease in the levels of neurotransmitters such as 5-hydroxytryptamine (5-HT), dopamine, and norepinephrine in the synaptic cleft of the brain. In addition, as a continuous chronic stress state, depression can cause continuous activation of the HPA axis and secretion of excessive glucocorticoids (GC), causing the hippocampus to be attacked by the excessive GC for a long time, and damaging the structure and function of hippocampal neurons. Hippocampal injury further aggravates neuroendocrine abnormalities and aggravates depression symptoms.

Based on the existing pathogenesis, the current antidepressants used in clinical practice mainly focus on increasing the level of monoamine neurotransmitters (mainly 5-HT) in the synaptic cleft of the brain. Among them, selective serotonin reuptake inhibitors (SSRI) and serotonin and norepinephrine reuptake inhibitors (SNRI) are the current first-line antidepressants, accounting for 70% or above of clinical drugs. However, the current clinical use of drugs has the following two problems. First, there is a latency of 2-4 weeks for the drugs to take effect. The reason is that after SSRI are taken to increase the 5-HT concentration in the synaptic cleft, 5-HT will counteract a pre-synaptic 5-HT1A receptor, resulting in negative feedback inhibition, and leading to a decrease in the synthesis and secretion of pre-synaptic 5-HT. After being taken continuously for 2-4 weeks and the 5-HT1A receptor is desensitized, SSRI can really take effect. However, these 2-4 weeks will lead to a decrease in patients' compliance with medication in clinical practice, and greatly increase the risk of suicide. Second, most antidepressants do not have a preventive effect, and long-term medication will produce side effects such as non-specific abdominal pain, constipation, diarrhea, indigestion, and gaseous distention. Therefore, exploring new antidepressant methods is particularly important, and also shows very broad market potential.

“Brain-gut axis” is a new concept proposed in recent years. As a two-way communication system between intestinal bacteria and the brain, the brain-gut axis mainly regulates the function and behavior of the brain through neural pathways, endocrine pathways and immune pathways. The intestine contains a large number (approximately 10¹⁴-10¹⁵) of intestinal microorganisms, and is the largest microecosystem in the human body. Normal communication between the intestinal flora and metabolites thereof and a host is necessary to maintain the health of the host. Disorders of the intestinal microecology are related to many diseases, including diabetes, obesity, inflammatory bowel disease, neurodegenerative diseases, tumors, etc. Regulation of the intestinal flora through probiotics has also become a new way to treat neurological diseases.

Therefore, screening out probiotics that can regulate the intestinal flora and effectively alleviate depression is very necessary, and is of great significance to deeply explore the functions of probiotics and develop probiotics with higher health value; and at the same time, it is beneficial to find out new ways and solutions to alleviate depression using dietary strategies.

### SUMMARY

The purpose of this section is to outline some aspects of the embodiments of the present invention and briefly introduce some preferred embodiments. Some simplifications or omissions may be made in this section and the abstract of the specification and invention title of this application to avoid obscuring the purpose of this section, the abstract of the specification and the name of the invention, and such simplification or omission cannot be used to limit the scope of the invention.

In view of the above-mentioned technical defects, the present invention has been proposed.

Therefore, as one aspect of the present invention, the present invention overcomes the deficiencies in the prior art and provides Bifidobacterium breve CCFM1025 (Bifidobacterium breve), which was deposited on Jun. 11, 2018 in the Guangdong Microbial Strain Collection The deposit address of the center is Guangdong Institute of Microbiology, 5th Floor, Building 59, Courtyard No. 100, Xianlie Middle Road, Guangzhou City, and the deposit number is GDMCC No. 60386.

As another aspect of the present invention, the present invention overcomes the deficiencies in the prior art and provides a fermented food.

In order to solve the above technical problems, the present invention provides the following technical solutions, wherein: the fermented food is produced by fermentation using Bifidobacterium breve CCFM1025, and the fermented food includes solid food, liquid food, and semi-solid food.

As a preferred method of the fermented food of the present invention, wherein: the fermented food includes dairy products, soy products, fruit and vegetable products, the dairy products include milk, sour cream, cheese; the fruit and vegetable products include Cucumber, carrot, beet, celery, cabbage products.

As another aspect of the present invention, the present invention overcomes the deficiencies in the prior art and provides the application of Bifidobacterium breve CCFM1025 in the preparation of in vivo colonized probiotic bacteria.

As another aspect of the present invention, the present invention overcomes the deficiencies in the prior art and provides the application of Bifidobacterium breve CCFM1025 in the preparation of antidepressant, anti-inflammatory bowel disease, anti-obesity drugs and health products.

As a preferred solution of the application of the Bifidobacterium breve CCFM1025 in the preparation of in vivo probiotics colonization, wherein: the Bifidobacterium breve CCFM1025 can improve the depression-like behavior of depressed mice and improve depression The levels of serotonin (5-HT), 5-hydroxytryptophan (5-HTP) and brain-derived neurotrophic factor (BDNF) in the mouse brain, and reduce the level of corticosterone in the serum of depressed mice; Bifidobacterium breve CCFM1025 can increase serum serotonin levels in depressed mice and improve gastrointestinal motility; Bifidobacterium breve CCFM1025 can improve intestinal flora disorder in depressed mice and reduce intestinal Weirong Abundance of Veillonellaceae, increase of abundance of Bifidobacterium and Allobaculum, increase intestinal flora α-diversity, reduce the occurrence of inflammatory bowel disease and obesity; the short Bifidobacterium can increase the mRNA level of tryptophan hydroxylase 1 in simulated intestinal chromaffin cells (RIN14B cells), and increase the secretion of 5-hydroxytryptophan from the cells, which can specifically stimulate the intestinal chromaffin Cells produce 5-hydroxytryptophan to provide precursors for the synthesis of serotonin in the brain.

As another aspect of the present invention, the present invention overcomes the deficiencies in the prior art and provides the fermented food according to claim 2 or 3 in the preparation of anti-depression, anti-inflammatory bowel disease, anti-obesity functional food Applications.

As a preferred solution of the application of the fermented food of the present invention in the preparation of anti-depressive functional food, wherein: the Bifidobacterium breve CCFM1025 can improve the depression-like behavior of depressed mice and improve depressed mice Levels of serotonin, 5-hydroxytryptophan and brain-derived neurotrophic factor in the brain, reduce corticosterone levels in the serum of depressed mice; the Bifidobacterium breve CCFM1025 can increase 5-Serotonin level improves gastrointestinal motility; Bifidobacterium breve CCFM1025 can improve the intestinal flora disorder in depressed mice, reduce the abundance of Veillonellaceae in the intestine, and increase the genus Bifidobacterium (Bifidobacterium) and Mycoplasma (Allobaculum) abundance, improve the intestinal flora α-diversity, reduce the incidence of inflammatory bowel disease and obesity; the Bifidobacterium breve can improve the intestinal chromaffin cells (RIN14B cells) Tryptophan hydroxylase 1 mRNA level, and increase the secretion of 5-hydroxytryptophan in this cell, can specifically produce 5-hydroxytryptophan by stimulating intestinal chromaffin cells to become 5-hydroxytryptamine in the brain The synthesis provides the precursor material. Beneficial effects of the disclosure: in an experiment of depressed model mice, taking CCFM1025 can significantly alleviate depression-like behaviors in the mice, and the assessment indicators include a forced swim test, a sucrose preference test and a step down test; taking CCFM1025 can significantly increase the levels of 5-hydroxytryptophane and 5-hydroxytryptamine in the hippocampus of the depressed mice, and at the same time significantly increase the level of BDNF in the prefrontal cortex; taking CCFM1025 can significantly reduce the level of corticosterone in the serum of the depressed mice and alleviate hyperfunction of HPA caused by depression; taking CCFM1025 can increase the level of 5-hydroxytryptamine in the serum tissue of the depressed mice and improve intestinal peristalsis; and taking CCFM1025 can improve intestinal flora disorders caused by depression, increase the α-diversity of the intestinal flora, reduce the abundance of Veillonellaceae in the intestine, increase the abundance of Bifidobacterium and Allobaculum, make the intestinal flora tend to normalize, and reduce the occurrence of inflammatory bowel disease and obesity. The results of in-vitro experiments show that CCFM1025 can increase the mRNA level of tryptophan hydroxylase 1 in simulated enterochromaffin cells (RIN14B cells), increase the secretion of 5-hydroxytryptophan in the cells, specifically stimulate the enterochromaffin cells to produce 5-hydroxytryptophane to provide a precursor substance for the synthesis of 5-hydroxytryptamine in the brain.

The B. breve CCFM1025 as described in the disclosure can be used to prepare food, health-care products and drugs with an antidepressant function, and has very wide application prospects.

Biomaterial Preservation

B. breve CCFM1025, with a category name of B. breve, was preserved at the Guangdong Microbial Culture Collection Center on Jun. 11, 2018, the preservation address is Guangdong Institute of Microbiology, 5th Floor, Building 59, Grand Courtyard 100, Xianlie Middle Road, Guangzhou, and the preservation number is GDMCC No. 60386.

### BRIEF DESCRIPTION OF FIGURES

In order to more clearly illustrate the technical schemes of the examples of the disclosure, the accompanying drawings used in the description of the examples are briefly described below. It is obvious that the accompanying drawings in the following description are only some examples of the disclosure, and other accompanying drawings may be obtained by those skilled in the art based on these accompanying drawings without any creative effort. In the figures:

FIG. 1 is a schematic diagram of the behavioral changes of mice after six weeks of strain intervention in depressed mice. (a) forced swimming experiment; (b) sugar water preference experiment; (c) platform jumping experiment; where *P<0.05, **P<0.01, ***P <0.001 (vs model group).

FIG. 2 Six weeks after this strain intervenes in depressed mice, serotonin (5-HT, panel a) and 5-hydroxytryptophan (5-HTP, panel b), prefrontal cortex in hippocampus tissue Schematic diagram of changes in the level of midbrain-derived neurotrophic factor (BDNF, Figure c); where *P<0.05, **P<0.01, ***P<0.001 (vs model group).

FIG. 3 is a schematic diagram of the level of corticosterone in the serum of mice, where **P<0.01, ***P<0.001 (vs. model group).

FIG. 4 is a schematic diagram of the level of 5-HT in the serum of mice, where **P<0.01 (vs. model group).

FIG. 5 is a schematic diagram of changes in α-diversity of intestinal flora of mice, where *P<0.05 (vs. model group).

FIG. 6 is a schematic diagram of changes in β-diversity of intestinal flora of mice.

FIG. 7 is a schematic diagram of changes in Veillonellaceae, Bifidobacterium, and Allobaculum in the intestines of mice, where *P<0.05, **P<0.01, ***P<0.001.

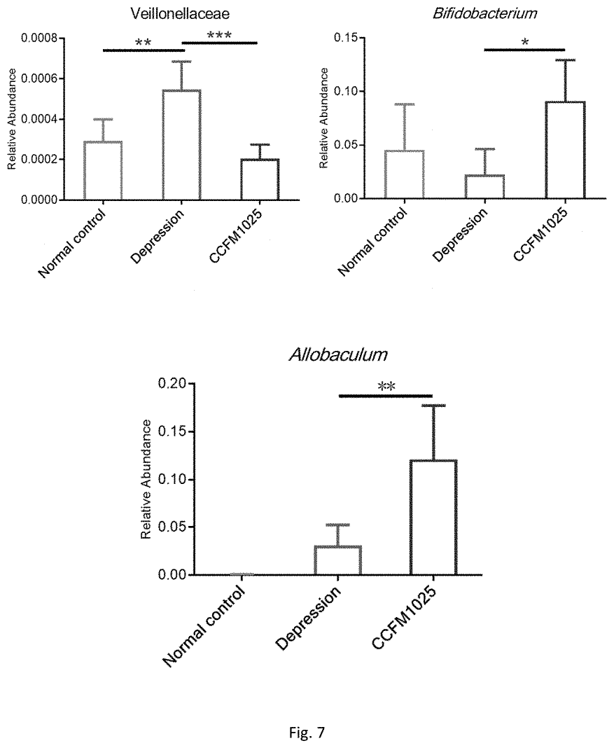

FIG. 8 is a schematic diagram showing changes in the mRNA level of tryptophan hydroxylase 1 in the cells and the content of 5-hydroxytryptophan in cell supernatant after RIN14B cells are stimulated by the strain, where *P<0.05, **P<0.01.

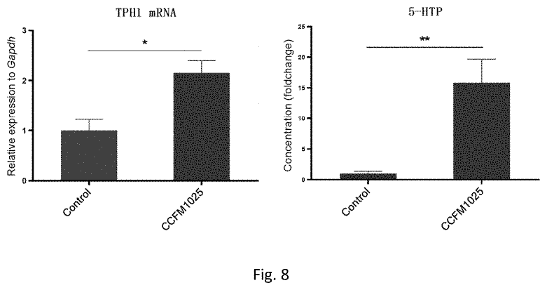

### DETAILED DESCRIPTION

For the purpose of making the objectives, characteristics and advantages of the disclosure clearer and more understandable, detailed description will be made to the specific implementations of the disclosure in conjunction with specific examples.

In the following description, many specific details are set forth in order to provide a full understanding of the disclosure. However, the disclosure may be implemented in other ways different from those described herein, and those skilled in the art can make similar promotion without violating the connotation of the disclosure. Therefore, the disclosure is not limited by the specific examples disclosed below.

In addition, “an example” or “examples” used herein refer to particular features, structures, or characteristics that are included in at least one implementation mode of the disclosure. “In an example” appearing in different places in this description is not intended to refer to the same example, nor is it a separate or selective example that is mutually exclusive with other examples.

The B. breve CCFM1025 has the Following Biological Characteristics:

(1) bacterial characteristics: gram-positive, non-spore-forming, and inactive;

(2) colony characteristics: small, milky white, round, neat, slightly convex, opaque, and moist and smooth in the surface;

(3) growth properties: the lowest growth temperature of the strain is 15° C. and the highest growth temperature is 45° C.; the best growth temperature is 35-37° C.; the optimum growth pH is 6.5; and the strain enters a stationary phase after being cultured for 18 h;

(4) the strain can significantly improve the behavioral manifestations of mice in depressed mouse models;

(5) the strain can increase the levels of 5-HT, 5-HTP and BDNF in the brain of the mice in the depressed mouse models;

(6) the strain can reduce the level of corticosterone in the serum of the mice in the depressed mouse models;

(7) the strain can increase the 5-HT level in the serum tissue of depressed mice, and improve the intestinal peristalsis thereof in the depressed mouse models; and

(8) the strain can reduce the abundance of Veillonellaceae in the intestine of the depressed mice, increase the abundance of Bifidobacterium and Allobaculum, increase the α-diversity of the intestinal flora, improve intestinal flora disorders caused by depression, and reduce the occurrence of inflammatory bowel disease and obesity.

An Extraction Method of the B. breve CCFM1025 Includes:

(I) Isolation and screening of Bifidobacterium:

(1) 1 g of fresh feces from a healthy adult is taken. After gradient dilution, the feces is spread on an mMRS solid medium and cultured in an anaerobic environment at 37° C. for 72 h.

(2) The colonial morphology is observed and recorded, and colonies are picked for purification by streaking.

(3) The colonies are cultured in an mMRS liquid medium at 37° C. for 48 h and then subjected to gram staining, and the colonial morphology is recorded.

(4) Gram-negative bacteria and gram-positive cocci are discarded from the colonies, and the gram-positive bacilli are selected.

(5) After catalase analysis, catalase-positive strains are discarded and catalase-negative strains are kept.

(II) Preliminary identification of Bifidobacterium: fructose-6-phosphate phosphoketolase assay

(1) The lactic acid bacteria screened in step (I) are cultured in a liquid mMRS medium for 24 h, and then 1 mL of the culture is taken and centrifuged at 8000 rpm for 2 min.

(2) The centrifugate is washed twice with a 0.05 M KH2PO4 solution with the pH of 6.5 containing 0.05% (mass percentage) of cysteine hydrochloride.

(3) The washed centrifugate is resuspended in 200 μL of a phosphate buffer containing 0.25% (mass percentage) of Triton X-100.

(4) 50 μL of a mixture of sodium fluoride with the concentration of 6 mg/mL and sodium iodoacetate with the concentration of 10 mg/mL and 50 μL of fructose-6-phosphate with the concentration of 80 mg/mL are added, and incubation is performed at 37° C. for 1 h.

(5) 300 μL of hydroxylamine hydrochloride with the concentration of 0.139 g/mL and the pH of 6.5 is added, and the mixture is placed at room temperature for 10 min.

(6) 200 μL of 15% (mass percentage) trichloroacetic acid and 4 M HCl are separately added.

(7) 200 μL of 0.1 M HCl containing 5% (mass percentage) of ferric chloride is added, the system quickly turns red, that is, the system is F6PPK positive, and the strain is preliminarily determined to be Bifidobacterium.

(III) Molecular Biological Identification of Bifidobacterium:

(1) Single-bacterial genome extraction: the Bifidobacterium screened in step (II) is cultured overnight. 1 mL of the bacterial suspension cultured overnight is taken in a 1.5 mL centrifuge tube and centrifuged at 10000 rpm for 2 min, and the supernatant is discarded to obtain bacteria. After being blow washed with 1 mL of sterile water, the bacteria are centrifuged at 10000 rpm for 2 min, and the supernatant is discarded to obtain the bacteria. 200 μL of SDS lysate is added, and subjected to water bath at 80° C. for 30 min. 200 μL of a phenol-chloroform solution is added to the bacterial lysate, where the components of the phenol-chloroform solution include Tris saturated phenol, chloroform and isoamyl alcohol in a volume ratio of Tris saturated phenol:chloroform:isoamyl alcohol=25:24:1. After being mixed upside down, the mixed solution is centrifuged at 12000 rpm for 5-10 min, and 200 μL of supernatant is taken. 400 μL of ice ethanol or ice isopropanol is added to the 200 μL of supernatant, enable the mixed solution to stand at −20° C. for 1 h, and centrifuged at 12000 rpm for 5-10 min, and the supernatant is discarded. 500 μL of 70% (volume percentage) ice ethanol is added to resuspend precipitates, the resuspension is centrifuged at 12000 rpm for 1-3 min, and the supernatant is discarded. The centrifugate is dried in an oven at 60° C., or air dried. 50 μL of ddH₂O is added to redissolve the precipitates for PCR.

(2) 16S rDNA PCR:

A. 50 μL PCR reaction system of 16S rDNA of bacteria:

10×Taq buffer, 5 μL; dNTP, 5 μL; 27F, 0.5 μL; 1492R, 0.5 μL; Taq enzyme, 0.5 μL; template, 0.5 μL; ddH₂O, 38 μL.

B. PCR conditions:

95° C., 5 min; 95° C., 10 s; 55° C., 30 s; 72° C., 30 s; steps 2-4, 30×; 72° C., 5 min; 12° C., 2 min.

C. 1% agarose gel is prepared, and then a PCR product is mixed with a 10000× loading buffer. The loading quantity of sample is 2 μL, agarose gel electrophoresis is performed for 30 min at 120 V, and then gel imaging is performed.

D. The obtained PCR product is sent to a professional sequencing company. The sequencing result (SEQ ID NO. 1) is subjected to search and similarity comparison in GeneBank using BLAST, and the strain is identified as B. breve.

(3) Whole Genome Sequencing

The extracted whole genome is sent to the professional sequencing company, and the whole genome of the bacteria is sequenced using a second-generation sequencer. The sequence result is subjected to search and similarity comparison in GenBank using BLAST, and the sequencing result is that the strain is identified as a newly discovered strain belonging to B. breve. The strain is preserved at −80° C. for later use.

### Example 1: B. breve CCFM1025 can Significantly Improve the Behavioral Indicators of Depressed Mice

32 6-week-old male C57BL/6J mice were selected. After one week of adaptation to the environment, the mice were randomly divided into four groups according to body weight: a control group, a model group, a fluoxetine intervention group, and a CCFM1025 intervention group, each containing 8 mice. Animal grouping and treatment methods are shown in Table 1.

TABLE 1
Animal experiment grouping and treatment methods

| | | Experimental |
|---|---|---|
| Group | Treatment method | period |
| Control | Free diet and water, gavage with a control | 6 weeks |
| group | solvent | |
| Model | Chronic unpredictable stress; | 6 weeks |
| group | free diet and water, gavage with a control | |
| | solvent | |
| Fluoxetine | Chronic unpredictable stress; | 6 weeks |
| intervention | free diet and water, gavage with 10 mg/kg | |
| group | fluoxetine | |
| CCFM1025 | Chronic unpredictable stress; | 6 weeks |
| intervention | free diet and water, gavage with 10⁹ CFU | |
| group | of live lactic acid bacteria | |

Chronic unpredictable stress depression mouse model: 1-2 kinds of stimuli were randomly performed every day, and the performance time of stimuli every day was determined randomly to avoid the circadian rhythm. Each method was used for not more than three times, and the stimuli were performed for five weeks. The stimuli include: (1) fasting for 24 h; (2) water deprivation+empty bottle stimulus for 24 h; (3) tail pinching for 3 min; (4) wet padding for 24 h; (5) immobilization for 1-2 h; (6) 450 tilted cage for 24 h; (7) continuous lighting for 24 h; (8) no padding for 24 h; (9) forced swim for 15 min; and (10) separation for 24 h.

Lactic acid bacteria gavage: the activated second generation of Bifidobacterium CCFM1025 was taken and cultured at 37° C. for 24 h, and the bacteria were collected after centrifugation at 4° C. and 8000 r/min for 3 min. The supernatant was discarded and the bacteria were resuspended with 5% sterilized skim milk to make the concentration of lactic acid bacteria reach 5×10⁹ CFU/mL. The gavage volume is 0.2 mL/mouse/day.

From the fifth week, the daily chronic unpredictable stress and intervention of drugs and probiotics were stopped, and behavioral tests were conducted on all mice, including forced swim test, tail suspension test, sucrose preference test, open field test and step down test. The specific implementation methods and results are as follows:

(1) Forced Swim Test:

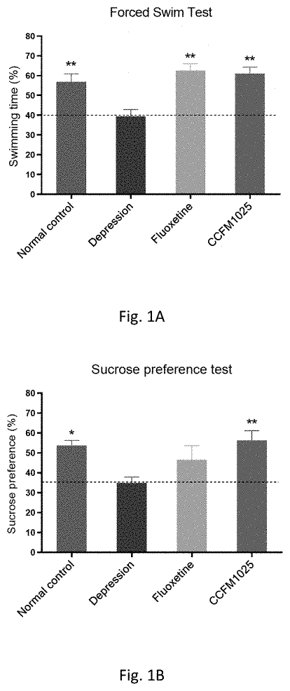

The forced swim test is a behavioral despair test, and is a classic test model for assessing antidepressant effects of drugs. A test bucket was filled with clean water about 20 cm high, and the water temperature was about 24±1° C. 24 h before the formal test, each mouse was subjected to a 15-minute swim training test. In the formal test, each mouse was tested for 6 min. The whole test was videoed using a camera to record the immobility (floating) time, that is, the limbs did not move or only the hind limbs moved slightly. As shown by the test results in FIG. 1A, the mice in the depression group had significantly reduced swimming time in the water, which shows a desperate behavior. Taking CCFM1025 could significantly improve this phenomenon, which indicates that the depression symptoms of the mice were alleviated.

(2) Sucrose Preference Test:

The sucrose preference test is a model for testing anhedonia in depression. Before starting the test, two identical drinking bottles were placed in a cage to allow the mice to drink adaptively for at least 3 days. After the adaptation, one of the bottles of water was replaced with an aqueous solution containing 1% sucrose. The intakes of water and sucrose solution were measured by weighing the bottles. The positions of the two bottles were changed every day to reduce drinking preferences due to different amounts of water. A formula for calculating sucrose preference is: sucrose preference=V(sucrose solution)/[V(sucrose solution)+V(water)]×100%. The test lasted for a total of 3 days, and the average value was taken. As shown by the test results in FIG. 1B, the depressed mice had a significant decrease in sucrose preference. After taking the CCFM1025, the mice restored their normal sucrose preference, which indicates that CCFM1025 could alleviate the anhedonia caused by depression.

(3) Step Down Test:

The first stage was an electric shock training stage. The mouse was put on a platform of a step down reaction box, an electric grid at the bottom of the reaction box was energized (38 V), and when stepping down the platform, the mouse was stimulated by an electric shock. The training lasted for 3 min. If any mouse did not step down the platform within 3 min, the mouse was driven off artificially to ensure that all animals were injured by the electric shock, resulting in nociceptive memory. After training, the mice were sent back into their original cages. The second stage (24 h after electric shock training) was a memory reproduction test stage. The mouse was put on the platform of the reaction box. The test lasted for 3 min and was not energized. The time from the mouse standing on the platform to the first step-down (the time staying on the platform), that is, the step down latency, was recorded. If the mouse did not step down the platform, the step down latency was calculated as 180 s. During the whole test, human interference with the animal behavior was avoided. The test results show (FIG. 1C) that the step down latency of depressed mice was significantly increased, which indicates an increase in depression and degree of anxiety, while the CCFM1025 could alleviate the degree of anxiety.

### Example 2: B. breve CCFM1025 can Significantly Increase the Levels of Neurotransmitters in the Brain of Depressed Mice

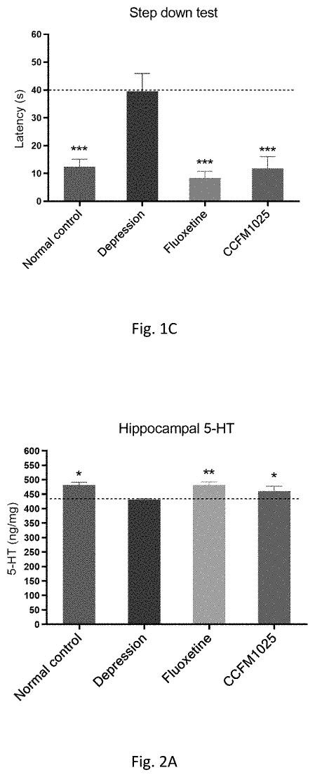

The mice in Example 2 were euthanized at the sixth weekend. The brain tissue of the mice was taken, and the hippocampus and the prefrontal cortex were separated on ice. A certain amount of fresh hippocampus and prefrontal cortex tissue (not less than 50 mg by weight) was separately taken. 9 times of a sterile PBS buffer by volume (equal to that 9 ml of homogenate was added to 1 g of tissue) was added, and the homogenate was homogenized with a tissue homogenizer. After the tissue fluid was centrifuged at 3000 g for 20 min, the supernatant was taken, and the contents of 5-HT and BDNF were measured with an ELISA kit. An equal volume of 5% perchloric acid was added to the supernatant of the hippocampus tissue fluid to precipitate protein, and the solution was centrifuged at 10000 g for 10 min. The supernatant was pipetted and filtered through a 0.22 μm water-based filter membrane, and then the content of 5-HTP was determined by the high performance liquid chromatography-fluorescence detection method (HPLC-FLD). A chromatographic column used is Shimadzu Intertsil ODS-3 (5 μm, 4.6 mm×250 mm). A mobile phase A is 0.1 mol/L NaAc (containing 0.1 mmol/L EDTA-2Na) with the pH of 5.1. A mobile phase B is methanol, the mobile phase A to the mobile phase B is equal to 85:15, the flow rate is 1.0 mL/min, the fluorescence detection excitation wavelength is 290 nm, the emission wavelength is 330 nm, and loading quantity of sample is 10 μL. The experimental results are shown in FIG. 2. The results show that taking CCFM1025 could significantly reverse the decrease in the levels of 5-HT and 5-HTP in the hippocampus and the level of BDNF in the prefrontal cortex caused by stress. Among them, CCFM1025 improved 5-HTP and 5-HT in the hippocampus to the same extent as fluoxetine, and the improvement effect on BDNF in the prefrontal cortex was significantly better than that of fluoxetine.

### Example 3: B. breve CCFM1025 can Reduce the Level of Corticosterone in the Serum of Mice

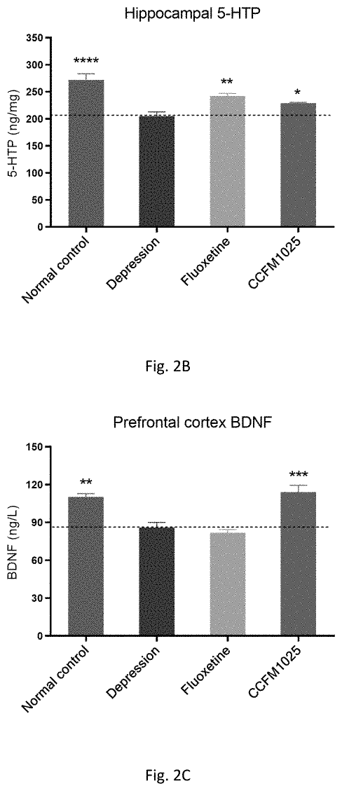

The mice in Example 2 were euthanized at the sixth weekend, and the blood of the mice was collected and centrifuged at 1000 g for 15 min to obtain serum. The content of corticosterone in the serum was measured using an ELISA kit. The experimental results (FIG. 3) show that due to continuous chronic stress, depressed mice had hyperfunction of the hypothalamic-pituitary-adrenal axis (HPA), and the level of corticosterone in the serum significantly increased. Taking the CCFM1025 could significantly reduce the level of corticosterone in the serum of the depressed mice, relieve HPA hyperactivity, and show good antidepressant effects.

### Example 4: B. breve CCFM1025 can Increase the Level of 5-HT in the Serum of Depressed Mice

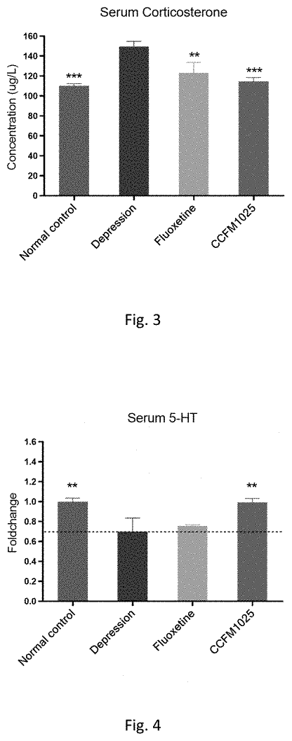

The serum collected in Example 3 was taken and the content of 5-HT in the serum was measured using an ELISA kit. 5-HT in peripheral tissue is of great significance for maintaining normal intestinal peristalsis. 5-HT in the peripheral tissue can stimulate 5-HT2 receptors in smooth muscles of the gastrointestinal tract, or act on 5-HT4 receptors in ganglion cells in the intestinal wall, causing contraction of the smooth muscles of the gastrointestinal tract, increasing gastrointestinal tension and accelerating intestinal peristalsis. The decrease in 5-HT in the peripheral tissue is directly related to diseases such as constipation and gastrointestinal discomfort. The experimental results (FIG. 4) show that 5-HT in the peripheral tissue of depressed mice significantly decreased, and intestinal peristalsis was impaired. Although taking fluoxetine has an antidepressant effect, fluoxetine cannot improve the intestinal function of the depressed mice. Taking CCFM1025 can not only alleviate the symptoms of depression, but also significantly increase and restore the content of 5-HT in the serum to normal, and restore normal intestinal peristalsis of the depressed mice.

### Example 5: B. breve CCFM1025 has a Regulating Effect on the Intestinal Flora of Depressed Mice and Multifunctional Regulating Effects Thereof

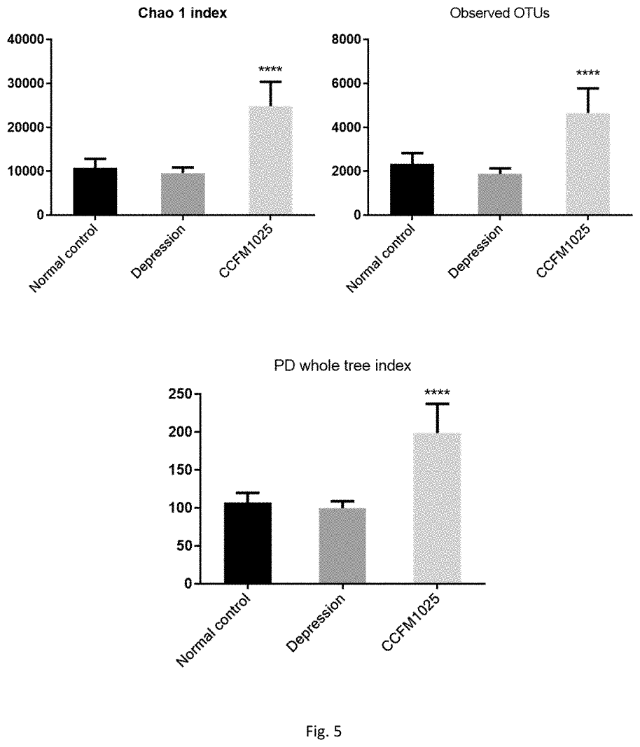

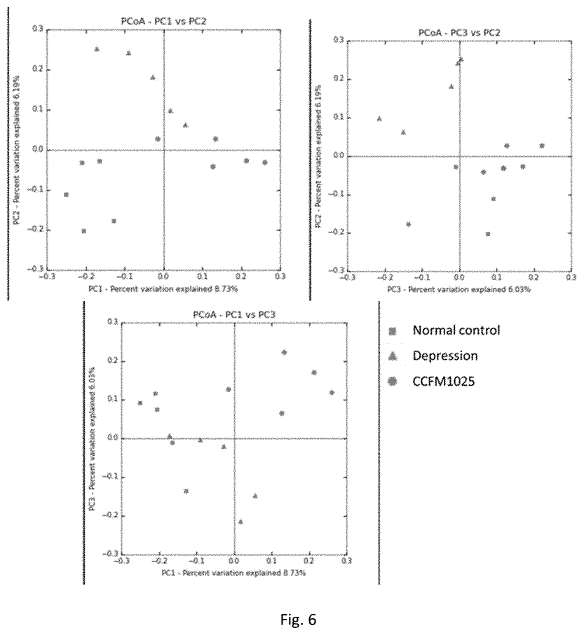

Fresh feces of the mice in Example 2 was taken at the sixth weekend, and total DNA was extracted from the mouse feces sample using an MP feces kit. Specific operation steps are as follows, mainly referring to the kit instructions. The mouse fecal genome was used as a template, V3-V4 fragments of 16S rDNA were amplified using a forward primer 520F (5′-AYTGGGYDTAAAGNG-3′) and a reverse primer 802R (5′-TACNVGGGTATCTAATCC-3′) as primers, and the length of a target fragment was about 247 bp. After the PCR reaction, all PCR samples in which target bands were observed were electrophoresed again, a 2.0% agarose gel was prepared, and electrophoresis was performed at 120 V for 40 min. After electrophoresis, the target bands were quickly cut under a UV lamp. The target band gel was extracted according to the instructions of a QIAquick Gel Extraction Kit. The DNA concentration of the sample was measured according to a Qubit DNA3.0 kit, then a library was constructed according to a TurSeq DNA LT Sample Preparation Kit and instructions thereof, and finally determination was performed on an Illumina Miseq sequencer according to a MiSeq Regent Kit and instructions thereof. After sequencing, sequences with the length smaller than 200 bp, primer sequences, and a single sequence that cannot be assembled were removed, and sequences were assembled according to a standard that overlapping bases are greater than 10 bp without mismatches. The sequences with the similarity greater than 97% were defined as a taxonomic unit (Operational Taxonomic Unit, OTU), and the species was determined by a Ribosomal Database Project (RDP) Naïve Bayesclassifier. The α-diversity and β-diversity of the sample were calculated to assess the bacterial diversity of the sample. Among them, the α-diversity is characterized by chao1 and PD whole tree indexes. The results (FIG. 5) show that the intestinal flora of depressed mice had decreased α-diversity, which indicates that depression was accompanied by a certain degree of intestinal flora disorder. Taking the CCFM1025 could significantly up-regulate the α-diversity of the intestinal flora and improve the species abundance of the intestinal flora. The β-diversity was assessed by principal coordinate analysis (PCoA) (FIG. 6). The results show that the intestinal flora of the depressed mice was significantly different from that of normal mice. After the mice took CCFM1025, their intestinal flora differed from that of the depression group to a certain extent, and had a tendency to transform to the normal mouse flora.

In addition, the abundance of Veillonellaceae significantly increased in the depressed mice, and taking CCFM1025 could significantly reverse this phenomenon. Veillonellaceae includes 6 genera and 25 species, and is Gram-negative, anaerobic or microaerobe cocci and coccobacilli. Some of the bacteria are conditioned pathogens, are common in human and animal microbial infections, and are also found in severe osteoarthritis and endocarditis. Taking CCFM1025 could also increase the abundance of Bifidobacterium and Allobaculum. Among them, Bifidobacterium has many important physiological functions such as biological barrier, nutrition, tumor resistance, immunity strengthening, gastrointestinal function improvement, and aging resistance, and is a physiological probiotic. Allobaculum can produce short-chain fatty acids, especially butyric acid, which can not only supply energy for oxidation of intestinal epithelial cells, but also has important effects of maintaining water electrolyte balance, regulating the balance of intestinal flora, regulating the intestinal barrier function, etc. In addition, short-chain fatty acids can exert anti-inflammatory effects through two signaling pathways: a G protein-coupled receptors (GPCRs) activation pathway, and a histone deacetylases (HDACs) inhibition pathway, and has a significant improvement effect on inflammatory bowel disease (IBD) and obesity. The results indicate that the CCFM1025 had multiple functions of regulating the intestinal flora, regulating immunity and intestinal barrier, and reducing the occurrence of inflammatory bowel disease and obesity in addition to the antidepressant function.

### Example 6: Effect of B. breve CCFM1025 on 5-HTP Synthesis in RIN14B Cells

Determination of 5-HTP in cell supernatant: RIN14B cells were inoculated in a 24-well plate at the density of 4×10⁵/mL and incubated for 72 h. The medium was discarded, the cells were washed with HBSS (1 mL) containing 0.1% bovine serum albumin (BSA) and 2 μM fluoxetine, and 1 mL of an HBSS suspension containing CCFM1025 was added (the control group used HBSS without bacteria). The cells were incubated at 37° C. for 20 min, the supernatant was collected and centrifuged at 6000 g for 5 min to remove precipitate, and the supernatant was frozen at −80° C. for testing. 5-HTP in the cell supernatant was measured by HPLC-FLD (referring to Example 3).

Determination of mRNA of TPH1: adherent cells in the above step were washed three times with HBSS, 1 mL of Trizol was added, and the cells were incubated on ice for 5-10 min. The cells were detached by blowing and patting, and the lysate was transferred to an enzyme-free EP tube. The total cell RNA was extracted by a conventional method, and cDNA synthesis was performed according to instructions of a reverse transcription kit (Prime Script RT reagent Kit gDNAEraser, Takara). The synthesized cDNA sample was tested for the concentration and purity (A260/A280) by an ultra-micro spectrophotometer (NanoDrop 2000C), and stored at −80° C. for later use. The sample was mixed with a fluorescent dye SYBR Green super mix (Qiagen, Germany). The PCR system contained 5 μL of mix, 1 μL of cDNA, and 1 μL of forward and reverse primers, and dd water was used to make up the total volume to 10 μL. Detection was performed on a real-time fluorescent quantitative gene amplification instrument CFX96™ Real-Time System (Bio-Rad, USA). 3 parallel holes were set up for each sample, and a housekeeping gene β-Actin was used as an internal reference. The results obtained were analyzed by a 2^{-ΔΔCq} method; and the primer sequences used are shown in Table 2.

TABLE 2
qPCR primer sequence

| Gene | Sequence | Sequence No. |
|---|---|---|
| TPH1 | Forward- | SEQ ID NO. 2 |
| | 5′-GCCTGTTACACA | |
| | TCGAGTCCC-3′ | |
| | Reverse- | SEQ ID NO. 3 |
| | 5′-ACAGTCTCCATA | |
| | ACGTCTTCCTT-3′ | |
| β-Actin | Forward- | SEQ ID NO. 4 |
| | 5′-CAGTCGGTTGGA | |
| | GCGAGCAT-3′ | |
| | Reverse- | SEQ ID NO. 5 |
| | 5′-GGACTTCCTGTA | |
| | ACAACGCATCT-3′ | |

The results show that after the CCFM1025 stimulated the RIN14B cells, the mRNA level of TPH1 in the cells was significantly increased. Correspondingly, the amount of 5-HTP secreted by the cells was also significantly increased. The 5-HTP secreted by the enterochromaffin cells can enter blood circulation and pass through a blood-brain barrier to provide a precursor substance for the synthesis of 5-HT in the brain. Therefore, the CCFM1025 can specifically stimulate the secretion of 5-HTP from enterochromaffin cells to promote the synthesis of 5-HT in the brain and achieve an antidepressant function.

### Example 7: Preparation of Fermented Food Containing the Bacteria Using the B. breve CCFM1025 of the Disclosure

Fresh vegetables were washed and juiced, and the juice was subjected to high-temperature instant sterilization. After high-temperature sterilization was performed at 140° C. for 2 s, the temperature was immediately reduced to 37° C., and the juice was inoculated with a B. breve CCFM1025 bacterial starter prepared by the disclosure to make the concentration reach 10⁶ CFU/mL or above, and was stored under refrigeration at 4° C., thereby obtaining a fruit and vegetable beverage containing the live B. breve CCFM1025 of the disclosure.

The disclosure can use the B. breve CCFM1025 to produce other fermented food by fermentation, including solid food, liquid food, and semi-solid food. The fermented food includes dairy products, bean products, and fruit and vegetable products; the dairy products include milk, sour cream, and cheese; and the fruit and vegetable products include cucumber, carrot, beet, celery, and cabbage products.

According to the methods of Examples 2 to 6, the effects of the fermented food prepared in Example 7 were respectively verified. The results show that the fermented food could improve the depression-like behaviors of depressed mice, increase the levels of 5-hydroxytryptamine, 5-hydroxytryptophane and brain-derived neurotrophic factors in the brains of the depressed mice, and reduce the level of corticosterone in the serum of the depressed mice; the fermented food could increase the level of 5-hydroxytryptamine in the serum of the depressed mice and improve gastrointestinal peristalsis; the fermented food could improve intestinal flora disorders in the depressed mice, reduce the abundance of Veillonellaceae in the intestine, increase the abundance of Bifidobacterium and Allobaculum, improve the α-diversity of intestinal flora, and reduce the occurrence of inflammatory bowel disease and obesity; and the fermented food could increase the mRNA level of tryptophan hydroxylase 1 in simulated enterochromaffin cells (RIN14B cells), increase the secretion of 5-hydroxytryptophan in the cells, and specifically stimulate the enterochromaffin cells to produce 5-hydroxytryptophan to provide a precursor substance for the synthesis of 5-hydroxytryptamine in the brain.

Although the disclosure has been disclosed as above in preferred examples, it is not intended to limit the disclosure. Anyone skilled in the art can make various changes and modifications without departing from the spirit and scope of the disclosure. Therefore, the protection scope of the disclosure should be defined by the claims.

1-8. (canceled)

9. A method for preventing or alleviating depression, resisting inflammatory bowel disease, and resisting obesity, wherein B. breve CCFM1025 or a probiotic preparation comprising the B. breve CCFM1025 is ingested into the body; and the B. breve CCFM1025 was preserved at the Guangdong Microbial Culture Collection Center on Jun. 11, 2018, the preservation address is Guangdong Institute of Microbiology, 5th Floor, Building 59, Grand Courtyard 100, Xianlie Middle Road, Guangzhou, and the preservation number is GDMCC No. 60386.

10. The method of claim 9, wherein the prevention or alleviation of depression comprises:
(a) alleviating depression-like behaviors of a depressed individual;
(b) increasing the levels of neurotransmitter 5-hydroxytryptamine and a neurotransmitter precursor 5-hydroxytryptophane in the brain;
(c) increasing the level of BDNF in the prefrontal cortex; and
(d) suppressing the level of corticosterone in the serum of the depressed individual.

11. The method of claim 9, wherein the resistance to obesity is specifically: reducing the abundance of Veillonellaceae in the intestine, increasing the abundance of Bifidobacterium and Allobaculum, increasing the α-diversity of intestinal microbiome, and reducing the occurrence of inflammatory bowel disease and obesity.

12. The method of claim 9, wherein the concentration of the B. breve CCFM1025 in the probiotic preparation is greater than or equal to 1×10⁵ CFU/mL or 1×10⁵ CFU/g.

13. The method of claim 9, wherein the B. breve CCFM1025 can be colonized in vivo.

14. Fermented food for preventing or alleviating depression, resisting inflammatory bowel disease and resisting obesity, wherein the fermented food is produced by fermentation using B. breve CCFM1025, and the fermented food comprises solid food, liquid food, and semi-solid food.

15. The fermented food of claim 14, wherein the fermented food comprises dairy products, bean products, and fruit and vegetable products; the dairy products comprise milk, sour cream, and cheese; and the fruit and vegetable products comprise cucumber, carrot, beet, celery, and cabbage products.

16. Application of B. breve CCFM1025 in preparing antidepressant, anti-inflammatory bowel disease, and anti-obesity drugs and health-care products.

17. The application of claim 16, wherein the drugs and health-care products have the following uses:
(a) improving depression-like behaviors of a depressed individual, and increasing the levels of 5-hydroxytryptamine, 5-hydroxytryptophane and brain-derived neurotrophic factors in brains of depressed mice;
(b) reducing the level of corticosterone in the serum of the depressed mice;
(c) increasing the level of 5-hydroxytryptamine in the serum of the depressed mice, and improving gastrointestinal peristalsis;
(d) improving intestinal microbiome disorders in the depressed mice, reducing the abundance of Veillonellaceae in the intestine, increasing the abundance of Bifidobacterium and Allobaculum, increasing the α-diversity of the intestinal microbiome, and reducing the occurrence of inflammatory bowel disease and obesity; and
(e) increasing the mRNA level of tryptophan hydroxylase 1 in simulated enterochromaffin cells, and increasing the secretion of 5-hydroxytryptophan in the cells.


SEQUENCE LISTING (5 sequences)

SEQ ID NO: 1 - DNA, 1344 bp, Bifidobacterium breve
caccggcttcgggtgctgcccactttcatgacttgacgggcggtgtgtacaaggcccgggaacgcattcaccgcgacgttgctgattcgcgattactagcgactccgccttcacgcagtcgagttgcagactgcgatccgaactgagaccggttttcagggatccgctccagctcgcgctgtcgcatcccgttgtaccggccattgtagcatgcgtgaagccctggacgtaaggggcatgatgatctgacgtcatccccaccttcctccgagttaaccccggcggtcccccgtgagttcccggcacaatccgctggcaacacggggcgagggttgcgctcgttgcgggacttaacccaacatctcacgacacgagctgacgacgaccatgcaccacctgtgaacccgccccgaagggaaaccccatctctgggatcgtcgggaacatgtcaagcccaggtaaggttcttcgcgttgcatcgaattaatccgcatgctccgccgcttgtgcgggcccccgtcaatttctttgagttttagccttgcggccgtactccccaggcgggatgcttaacgcgttagctccgacacggaacccgtggaacgggccccacatccagcatccaccgtttacggcgtggactaccagggtatctaatcctgttcgctccccacgctttcgctcctcagcgtcagtaacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataattccggataacgcttgcaccctacgtattaccgcggctgctggcacgtagttagccggtgcttattcgaaaggtacactcaacacaaaatgccttgctccctaacaaaagaggtttacaacccgaaggcctccatccctcacgcggcgtcgctgcatcaggcttgcgcccattgtgcaatattccccactgctgcctcccgtaggagtctgggccgtatctcagtcccaatgtggccggtcgccctctcaggccggctacccgtcgaagccatggtgggccgttaccccgccatcaagctgataggacgcgaccccatcccatgccgcaaaggctttcccaacacaccatgcagtgtgatggagcatccggcattaccacccgtttccaggagctattccggtgcatggggcaggtcggtcacgcattactcacccgttcgccactctcaccaccaagca

SEQ ID NO: 2 - DNA, 21 bp, Artificial sequence
  Synthetic DNA
gcctgttacacatcgagtccc

SEQ ID NO: 3 - DNA, 23 bp, Artificial sequence
  Synthetic DNA
acagtctccataacgtcttcctt

SEQ ID NO: 4 - DNA, 20 bp, Artificial sequence
  Synthetic DNA
cagtcggttggagcgagcat

SEQ ID NO: 5 - DNA, 23 bp, Artificial sequence
  Synthetic DNA
ggacttcctgtaacaacgcatct